(19) United States
(12) Patent Application Publication
(10) Pub. No.: US 2025/0001073 A1
(43) Pub. Date: Jan. 2, 2025

(54) INSERTER DEVICES PROVIDING MANUAL INSERTION AND AUTOMATIC RETRACTION

(71) Applicant: Eli Lilly and Company, Indianapolis, IN (US)

(72) Inventor: Mary Teresa CARTER, Wilmington, MA (US)

(21) Appl. No.: 18/697,785

(22) PCT Filed: Oct. 26, 2022
(86) PCT No.: PCT/US2022/047837

§ 371 Date: Apr. 2, 2024

Related U.S. Application Data
(60) Provisional application No. 63/273,249, filed on Oct. 29, 2021.

(51) Int. Cl. A61M 5/158

(52) U.S. Cl. CPC A61M 5/158; A61M 2005/1585; A61M 2005/1586

(57) ABSTRACT

Inserter devices provide manual insertion of an insertion needle and cannula and subsequent automatic retraction of the insertion needle. The inserter devices include insertion guards that inhibit insertion of the insertion needle and cannula unless a compressive force applied to the device exceeds a threshold. As the user applies more compressive force, the device remains unactuated until the threshold-force is exceeded, whereupon the movement is sudden and rapid. As a result, it feels to the user that the insertion is being driven by an automatic insertion mechanism.

### FIELD OF THE DISCLOSURE

The present disclosure relates to drug infusion systems, more specifically an inserter device that can be used to insert a cannula into the subcutaneous space of a patient, and methods of using the same.

### BACKGROUND

Infusion sets are used to deliver a drug to the subcutaneous space of a patient. The head assembly of the infusion set has a fluid path in the form of a stainless-steel needle or soft cannula that must be inserted to the correct depth in the subcutaneous tissue. To insert a soft cannula, a stainless-steel insertion needle is used. The insertion needle opens a hole in the tissue to allow the cannula to enter and provides stiffness for the cannula as it is inserted. After insertion, the insertion needle is removed.

The insertion and retraction of the insertion needle constitute separate steps which may be performed manually by a user or automated through an inserter (that is, the inserter providing one or more forces to move the components thereof). Most commercially available inserters automate insertion of the insertion needle. After insertion, the insertion needle is typically retracted manually.

Another important consideration is which components move during insertion. Most inserters are “shift head” devices 1000 (FIG. 1), in which the fluid path 1002 is permanently coupled to the infusion head 1004 and travels with the infusion head 1004 during the insertion process as an integrated infusion head assembly 1006. In contrast, other inserters are “shift fluid path” devices 2000 (FIG. 2), in which the fluid path 2002 is initially separated from the infusion head 2004 and is advanced toward and joined to the infusion head 2004 during the insertion process.

Shift head devices 1000 illustrated in FIG. 1 and shift fluid path devices 2000 illustrated in FIG. 2 each have various advantages over each other. For example, shift head devices 1000 of FIG. 1 may be more mechanically robust because the entire infusion head assembly 1006 moves together. Shifting the entire infusion head assembly 1006 also may facilitate omitting gaps between components that might allow water ingress between them, which in turn reduces infection risks. In contrast, for shift fluid path devices 2000 (FIG. 2), the infusion head 2004 and an adhesive patch are initially attached to the patient's skin when the device 2000 is positioned on the skin. When the patient or the patient's caregiver activates the device 2000, the fluid path 2002 shifts towards the skin and couples to the infusion head 2004. Because the adhesive patch and infusion head 2004 are initially attached to the skin, shift fluid path devices 2000 may be smaller and more compact. However, some such devices may have additional mechanical complexity due to the increased number of components and the dynamic interface. Coupling the fluid path 2002 into the infusion head 2004 must also be controlled to minimize unwanted crevices near the insertion site. Clips 2006 of FIG. 2 that capture the fluid path 2002 may have some tolerance to them, so that—even if captured correctly—the fluid path 2002 may wobble slightly within the clips, potentially causing discomfort.

Some commercially-available inserters provide manual insertion of the insertion needle, and after insertion, automatically retract the insertion needle. During the insertion process, the user may vary the force applied to the inserter to control the speed of insertion and reduce pain. However, some users prefer to avoid monitoring needle insertion and adjusting the force applied to the inserter. Accordingly, improved inserter devices are needed.

### SUMMARY

The present disclosure relates to inserter devices that provide manual insertion of an insertion needle and cannula and subsequent automatic retraction of the insertion needle. The inserter devices include insertion guards that inhibit insertion of the insertion needle and cannula unless a compressive force applied to the device exceeds a threshold. In some embodiments, as a user applies more compressive force, once the threshold-force is exceeded, the movement is sudden and rapid. As a result, in these embodiments it may feel to the user that the insertion is being driven by an automatic insertion mechanism.

According to an exemplary embodiment of the present disclosure, an inserter device is disclosed including a base; an actuator movably coupled to the base; a push plate coupled to the actuator and movable with the actuator relative to the base; an infusion cannula detachably coupled to the push plate and configured to be inserted into a subcutaneous space of a patient; an insertion needle coupled to the push plate and the infusion cannula; and a guard. The guard (1) retains the push plate, the insertion needle, and the infusion cannula in a stowed configuration in the base when a force applied to the actuator is less than a threshold; and (2) permits the push plate, the insertion needle, and the infusion cannula to move from the stowed configuration to an insertion configuration in which the infusion cannula and the insertion needle extend from the base when the force applied to the actuator is equal to or greater than the threshold.

According to another exemplary embodiment of the present disclosure, an inserter device is disclosed including a base; an actuator movably coupled to the base; a push plate coupled to the actuator and movable with the actuator relative to the base; an infusion cannula detachably coupled to the push plate and configured to be inserted into a subcutaneous space of a patient; an insertion needle coupled to the push plate and the infusion cannula; and a guard. The guard (1) inhibits movement of the actuator, the push plate, the insertion needle, and the infusion cannula relative to the base when a force applied to the actuator is less than a threshold; and (2) permits movement of the actuator, the push plate, the insertion needle, and the infusion cannula relative to the base when the force applied to the actuator is equal to or greater than the threshold, the push plate and the insertion needle thereby inserting the infusion cannula into the subcutaneous space of the patient.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a schematic view of a prior art example of a shift head inserter device.

FIG. 2 is a schematic view of a prior art example of a shift fluid path inserter device.

FIG. 3 is a perspective view of a shift head inserter device according to an embodiment of the present disclosure.

FIG. 4 is a side transverse sectional view of the inserter device along line 4-4 of FIG. 3.

FIG. 5 is a front transverse sectional view of the inserter device along line 5-5 of FIG. 3.

FIG. 6 is an exploded view of the inserter device of FIG. 3.

FIG. 7 is a rear transverse sectional view of the inserter device of FIG. 3 being moved from a stowed configuration toward an insertion configuration.

FIG. 8 is a detail view of the area of the inserter device within line 8-8 on FIG. 7.

FIG. 9 is a side transverse sectional view of the inserter device of FIG. 3 being moved from the stowed configuration toward the insertion configuration.

FIG. 10 is a side transverse sectional view of the inserter device of FIG. 3 upon reaching the insertion configuration.

FIG. 11 is a front transverse sectional view of the inserter device of FIG. 3 upon reaching the insertion configuration.

FIG. 12 is a side transverse sectional view of the inserter device of FIG. 3 upon reaching a retraction configuration.

FIG. 13 is a front transverse sectional view of the inserter device of FIG. 3 being detached from an infusion head assembly.

FIG. 14 is a perspective view of a shift fluid path inserter device according to another embodiment of the present disclosure.

FIG. 15 is a front transverse sectional view of the inserter device along line 15-15 of FIG. 14.

FIG. 16 is another front transverse sectional view of the inserter device along line 16-16 of FIG. 14.

FIG. 17 is an exploded view of the inserter device of FIG. 14.

FIG. 18 is a front transverse sectional view of the inserter device of FIG. 14 being moved from a stowed configuration toward an insertion configuration.

FIG. 19 is a side transverse sectional view of the inserter device of FIG. 14 being moved from the stowed configuration toward the insertion configuration.

FIG. 20 is an enlarged front transverse sectional view of the inserter device of FIG. 14 being moved further toward the insertion configuration.

FIG. 21 is an enlarged front transverse sectional view of the inserter device of FIG. 14 upon reaching the insertion configuration.

FIG. 22 is a rear transverse sectional view of the inserter device of FIG. 14 upon reaching the insertion configuration.

FIG. 23 is a side transverse sectional view of the inserter device of FIG. 14 upon reaching the insertion configuration.

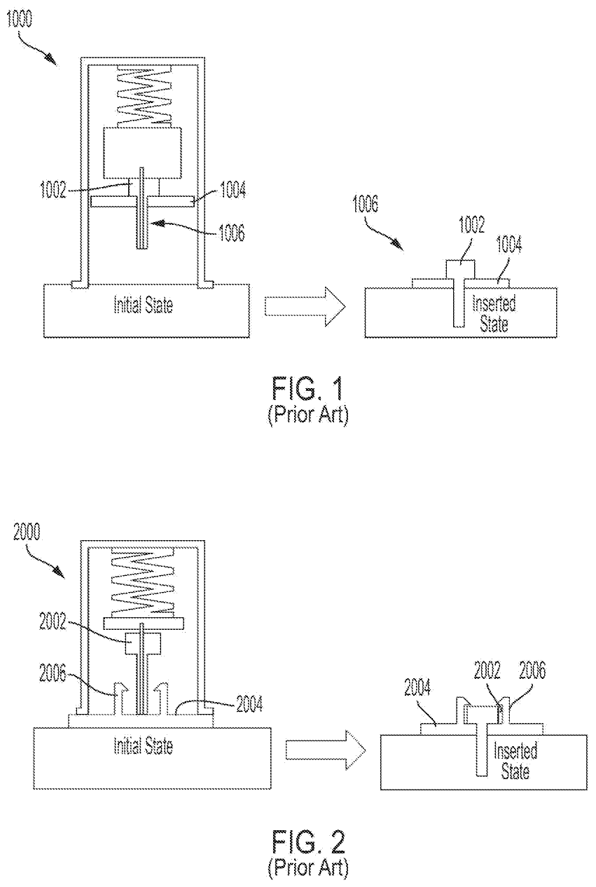

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

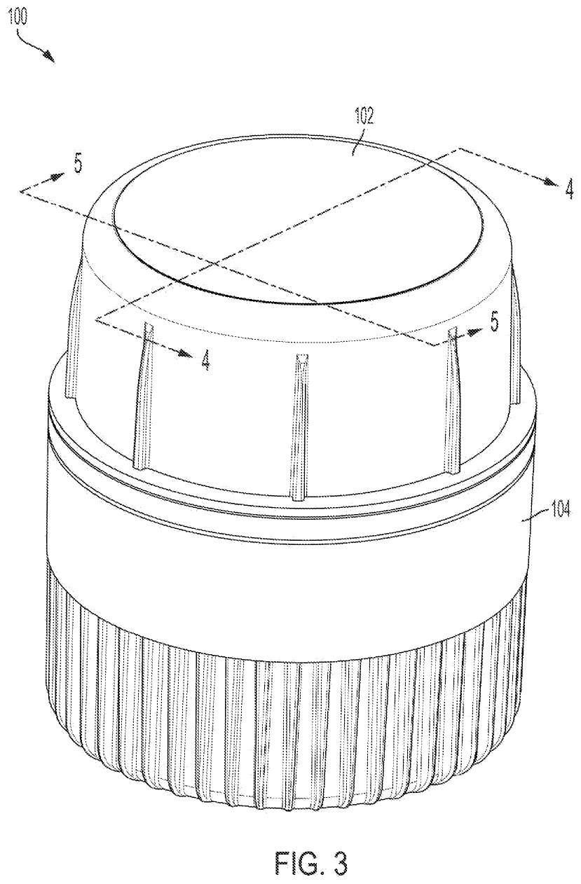

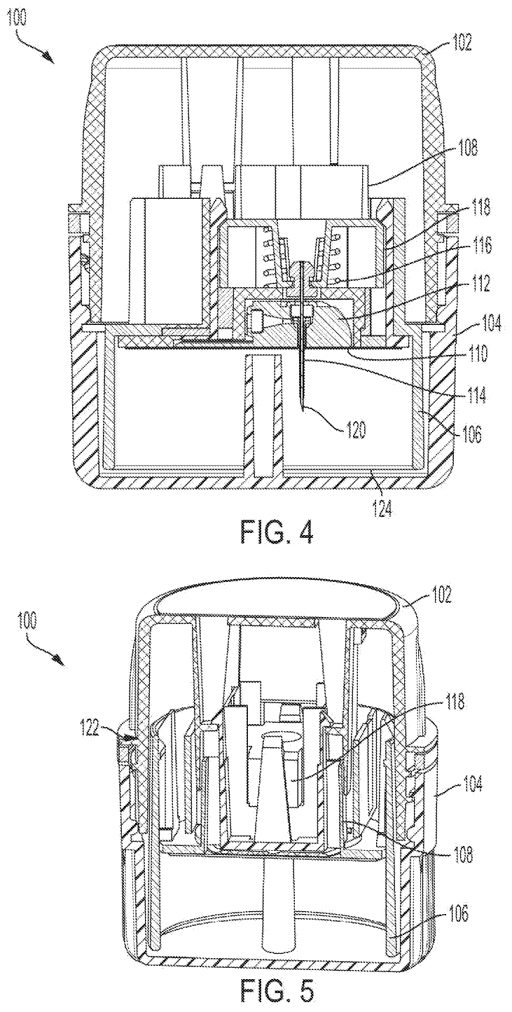

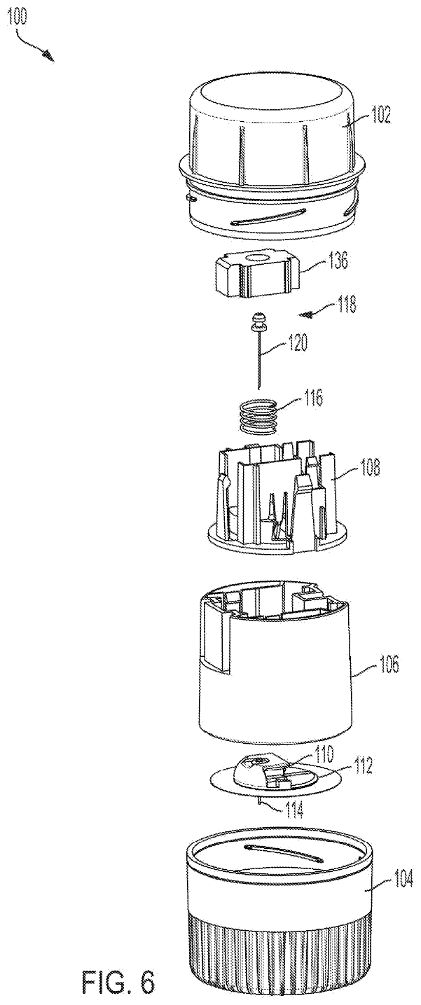

FIGS. 3-6 illustrate a shift head inserter device 100 according to an exemplary embodiment of the present disclosure. The inserter device 100 generally includes an actuator 102, which may also be referred to as a button, that detachably couples to a cap 104. With specific reference to FIGS. 4-6, internally the inserter device 100 includes a base 106 that movably supports a push plate 108. The actuator 102 and the push plate 108 are movable together relative to the base 106. The push plate 108 detachably supports an infusion head assembly 110 that includes an infusion head 112 with an associated adhesive patch (shown elsewhere) and an infusion cannula 114. As described in further detail below, the infusion head assembly 110 is configured to be attached to the skin of a patient such that the infusion cannula 114 is inserted in the subcutaneous space of the patient. The push plate 108 also couples to a retraction mechanism 116, such as a compression spring, and a needle assembly 118. The needle assembly 118 includes an insertion needle 120 and a needle carrier 136. The insertion needle 120 of the needle assembly 118 initially extends through the cannula 114 and, as described in further detail herein, the insertion needle 120 pierces the skin of the user to facilitate inserting the infusion cannula 114 in the subcutaneous space of the patient.

As described in further detail below, the inserter device 100 also includes guards that inhibit movement of one or more of the above components unless certain conditions are met. For example, the device 100 includes an insertion guard, shown indicatively in location 122 of FIG. 5, that inhibits insertion of the insertion needle 120 and the cannula 114 unless a compressive force applied to the actuator 102 exceeds a threshold. As a result, the device 100 appears, as viewed by a patient, to provide automatic insertion.

A method of using the inserter device 100 to attach the infusion head assembly 110 to the skin of a patient is described in further detail below. Before using the device 100 according to this method, however, the cap 104 is detached from the button 102, for example, by unscrewing the cap 104 from the button 102. The inserter device 100 thereby occupies a ready state. In the ready state, the push plate 108 and the infusion head assembly 110, including the insertion needle 120, and the infusion cannula 114 are disposed in a stowed configuration in the base 106. The insertion needle 120, which is attached to the needle carrier 136, penetrates through a top septum of the infusion head assembly 110 and is disposed within and concentric to the soft cannula 114. More specifically and as shown in FIG. 4, the tip of the insertion needle 120 is disposed within the base 106 and proximally relative to the distal end 124 of the base 106 (or, as shown FIG. 4, above the distal end 124 of the base 106). The tip of the insertion needle 120 is disposed apart from the distal end 124 of the base 106 by a sufficient distance to inhibit the tip of the needle 120 from contacting the patient prior to activation of the device 100, but not so large that the device 100 is unduly tall. In some embodiments, this distance is preferably between 5 and 10 mm.

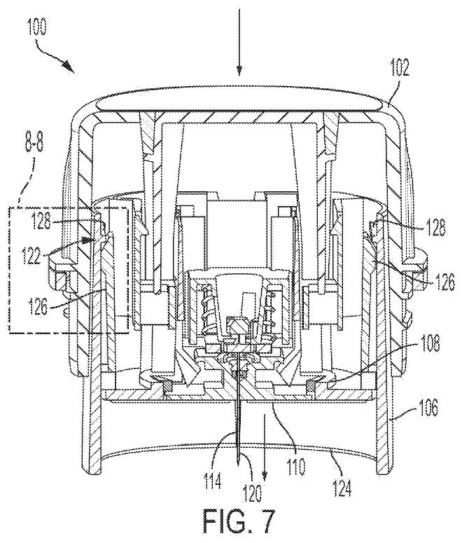

FIGS. 7-13 illustrate the method of using the inserter device 100 to attach the infusion head assembly 110 to the skin of the patient (not shown). First, when the inserter device 100 is in the ready state, the base 106 is held against the skin of the patient such that the distal end 124 of the base 106 is disposed adjacent the skin of the patient. Next and referring FIG. 7, a user (for example, the patient, a medical practitioner, or another person) applies a compressive force to the actuator 102 such that the actuator 102 and the push plate 108 are urged toward the base 106 and the skin of the patient. If the force is less than a threshold (for example, 10 Newtons), the insertion guard 122 holds the push plate 108, the infusion head assembly 110, the needle assembly 118 in the stowed configuration. However, and as shown in FIGS. 7-9, if the force is equal to or greater than the threshold, the insertion guard 122 permits the push plate 108, the infusion head assembly 110, the insertion needle 120, and needle hub 118 to move from the stowed configuration toward an insertion configuration.

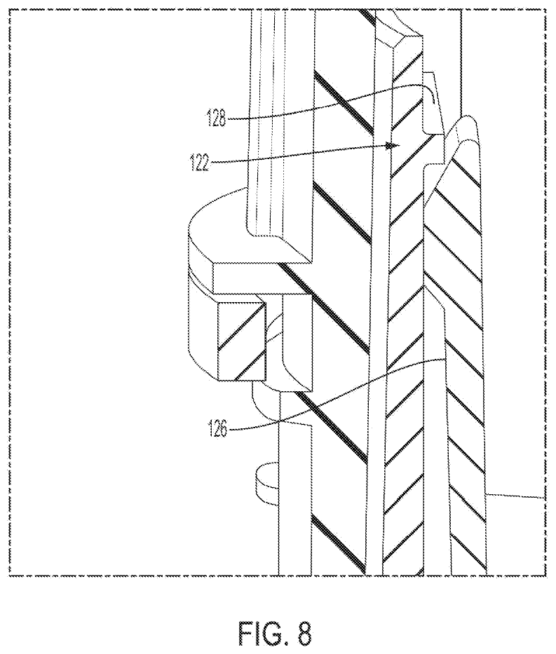

Illustratively, the insertion guard 122 includes one or more flexible snaps or arms 126 (more specifically, two arms 126) of the push plate 108 that each contact a ledge 128 of the base 106. When the force is equal to or greater than the threshold, the arms 126 deflect and slide over the ledges 128, as shown in FIGS. 7 and 8, to permit the device 100 to move from the stowed configuration toward the insertion configuration. In other embodiments, the insertion guard 122 may take other forms.

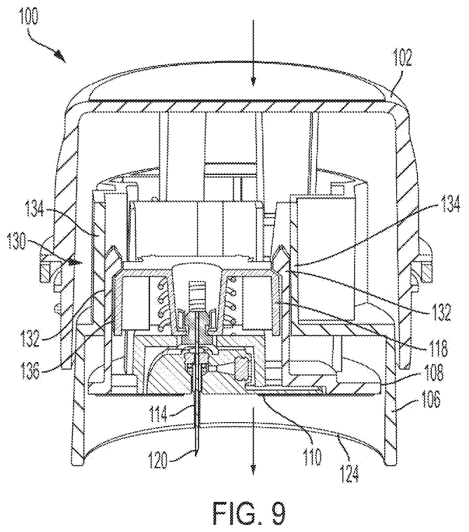

With specific reference to FIG. 9, when the push plate 108 moves from the stowed configuration toward the insertion configuration, the needle assembly 118 moves with the push plate 108 due to the presence of a retraction guard 130. Illustratively, the retraction guard 130 includes one or more flexible snaps or arms 132 (more specifically, two arms 132) of the push plate 108 that each slidably contact a retainer wall 134 of the base 106. The retainer walls 134 hold the arms 132 in contact with the needle carrier 136 of the needle assembly 118 and thereby cause the needle assembly 118 to move with the push plate 108 from the stowed configuration toward the insertion configuration. In other embodiments, the retraction guard 130 may take other forms.

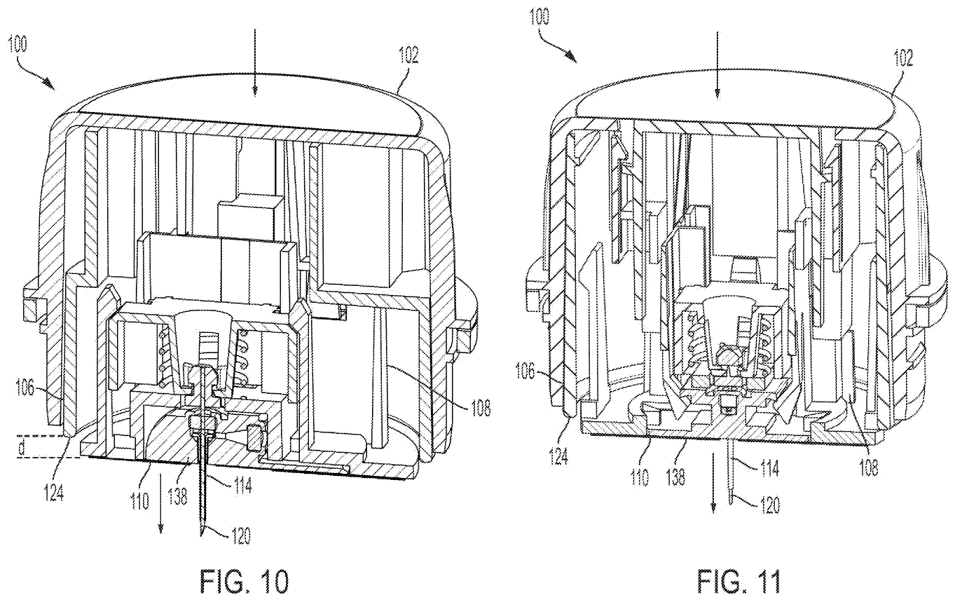

As shown in FIGS. 10 and 11, continued application of a compressive force to the actuator 102 causes the inserter device 100 to reach the insertion configuration. In the insertion configuration, the insertion needle 120 and the infusion cannula 114 extend outwardly from the distal end 124 of the base 106. As a result, the insertion needle 120 pierces the skin of the patient and the infusion cannula 114 enters the subcutaneous space of the patient. In the insertion configuration, the push plate 108 may extend a distance d below the distal end 124 of the base 106 (for example, 1 to 2 mm). This facilitates compressing the skin of the patient and securing the infusion head assembly 110 to the skin via an adhesive patch or surface 138 carried on the distal surface of the infusion head assembly 110. In some embodiments, the adhesive surface 138 is the only adhesive surface of the device 100. More specifically and advantageously, the device 100 lacks a second adhesive surface or layer that is initially secured to the skin of the patient and subsequently secured to the infusion head assembly 110 via the adhesive surface 138.

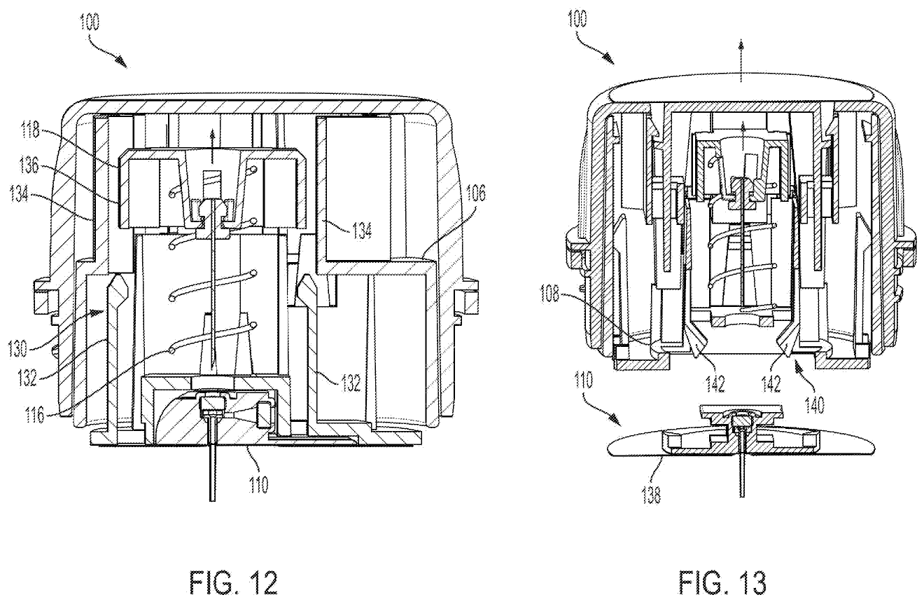

As shown in FIG. 12, after reaching the insertion configuration the retraction guard 130 disengages and permits the retraction mechanism 116 to automatically move the needle assembly 118 from the insertion configuration to a retracted configuration in the base 106. More specifically, the arms 132 of the retraction guard 130 disengage the retainer walls 134 of the base 106, and the arms 132 are deflected outwardly by expansion of the retraction mechanism 116 and proximal movement of the needle assembly 118 relative to the base 106. As a result, the needle carrier 136 slides over the arms 132, and the needle assembly 118 moves from the insertion configuration to the retracted configuration. The infusion head assembly 110, however, remains attached to the skin of the patient.

As shown in FIG. 13, the inserter device 100 is then moved away from the skin of the patient to leave the infusion head assembly 110 secured to the skin of the patient. More specifically, a deployment guard 140, which initially held the infusion head assembly 110 to the push plate 108, disengages to permit the infusion head assembly 110 to detach from the push plate 108. Illustratively, the deployment guard 140 includes one or more snaps or arms 142 (for example, two arms 142) that initially contact the infusion head assembly 110. The arms 142 provide a relatively low force for holding the infusion head assembly 110 to the push plate 108 compared to the force provided by the adhesive surface 138 for holding the infusion head assembly 110 to the skin of the patient. As a result, the arms 142 deflect and the infusion head assembly 110 slides thereover when the inserter device 100 is moved away from the skin of the patient, and the infusion head assembly 110 remains secured to the skin of the patient. In other embodiments, the deployment guard 140 may take other forms. Finally, the “empty” inserter device 100 may be discarded, and the infusion head assembly 110 may be used to deliver a drug to the patient.

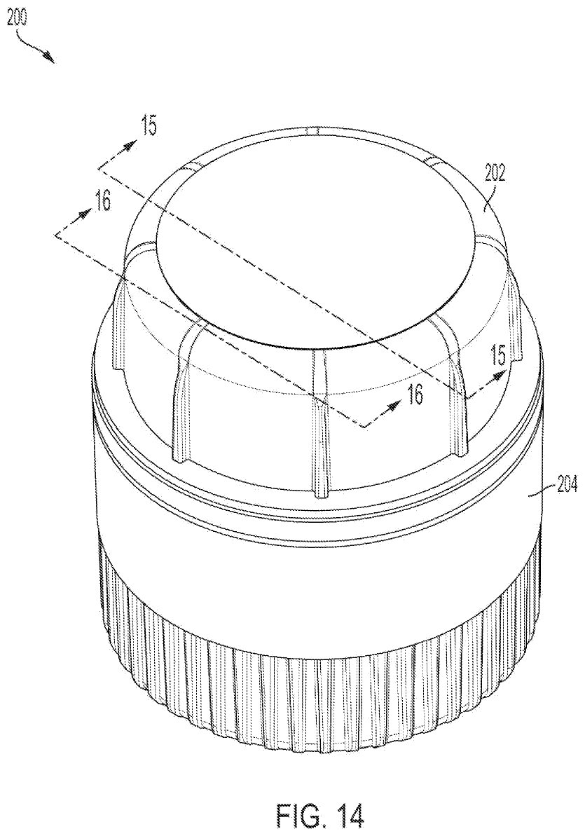

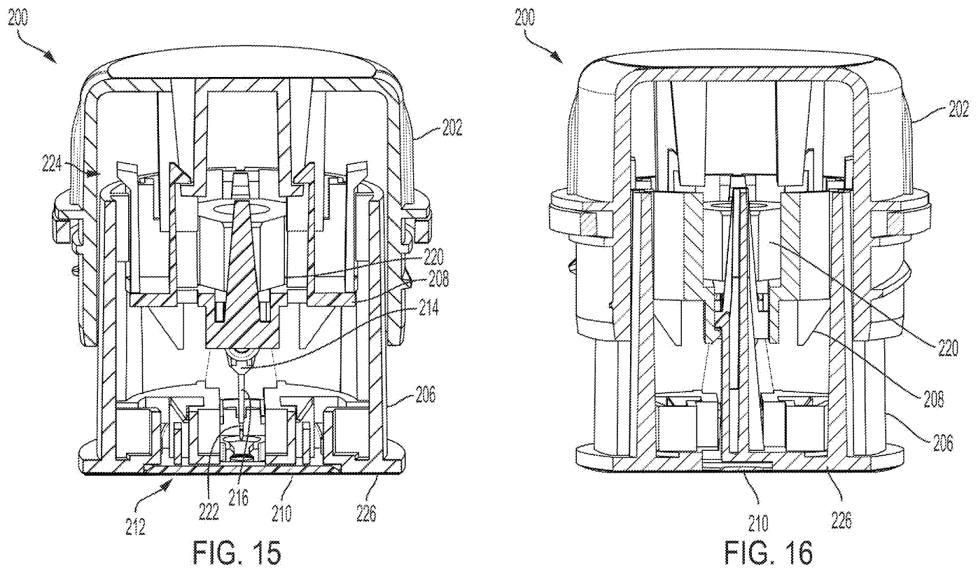

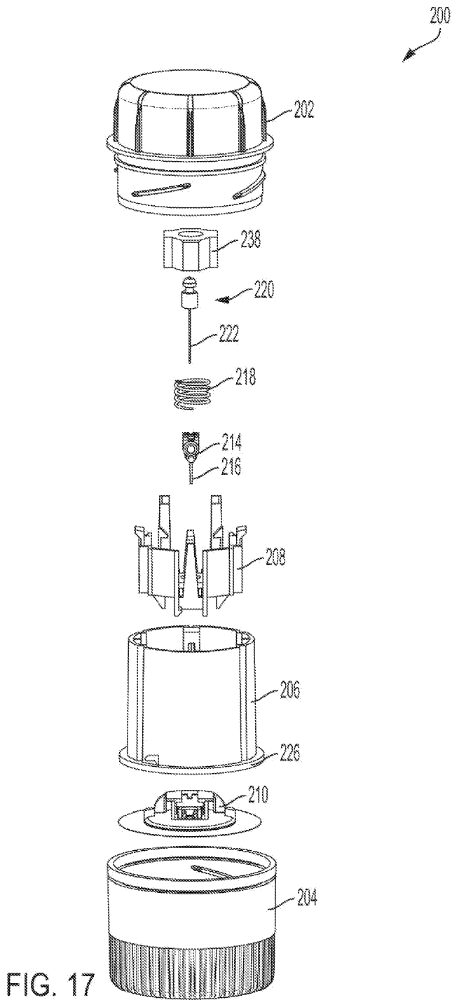

FIGS. 14-17 illustrate a shift fluid path inserter device 200 according to another exemplary embodiment of the present disclosure. The inserter device 200 generally includes similar components to those of the inserter device 100. More specifically, the inserter device 200 includes an actuator 202 that detachably couples to a cap 204. With specific reference to FIGS. 15-17, internally the inserter device 200 includes a base 206 that movably supports a push plate 208. The actuator 202 and the push plate 208 are movable together relative to the base 206. Unlike the components of the device 100, the base 206 initially detachably supports an infusion head 210 of an infusion head assembly 212 (FIG. 15) while the push plate 208 initially detachably supports a cannula assembly 214 of the infusion head assembly 212. As described in further detail below, the infusion head 210 is configured to be attached to the skin of a patient, and the cannula assembly 214 is configured to be attached to the infusion head 210 such that an infusion cannula 216 of the cannula assembly 214 is inserted in the subcutaneous space of the patient. The push plate 208 also couples to a retraction mechanism 218 (FIG. 17), such as a compression spring, and a needle assembly 220 that includes an insertion needle 222 and a needle carrier 238. The insertion needle 222 of the needle assembly 220 initially extends through the cannula 216 and, as described in further detail below, the insertion needle 222 pierces the skin of the user to facilitate inserting the cannula 216 in the subcutaneous space of the patient.

Like the inserter device 100 and as described in further detail below, the inserter device 200 also includes guards that inhibit movement of one or more of the above components unless certain conditions are met. For example, the device 200 includes an insertion guard, shown indicatively at location 224 (FIG. 15) that inhibits insertion of the insertion needle 222 and the cannula 216 unless a compressive force applied to the actuator 202 exceeds a threshold. As the user applies more compressive force, the device 200 is not actuated until the threshold-force is exceeded, whereupon the movement is sudden and rapid. As a result, it feels to the user that the insertion is being driven by an automatic insertion mechanism—using stored energy such as that found in a spring.

A method of using the inserter device 200 to attach the infusion head assembly 212 to the skin of a patient is described in further detail below. Before using the device 200 according to this method, however, the cap 204 is detached from the base 206, for example, by unscrewing the cap 204 from the base 206. The inserter device 200 thereby occupies a ready state. In the ready state, the push plate 208, the insertion needle assembly 220, and the cannula assembly 214 are disposed in a stowed configuration in the base 206. More specifically and as shown in FIG. 15, the insertion needle assembly 220 and the cannula assembly 214 are disposed within the base 206 and proximally relative to a distal end 226 of the base 206.

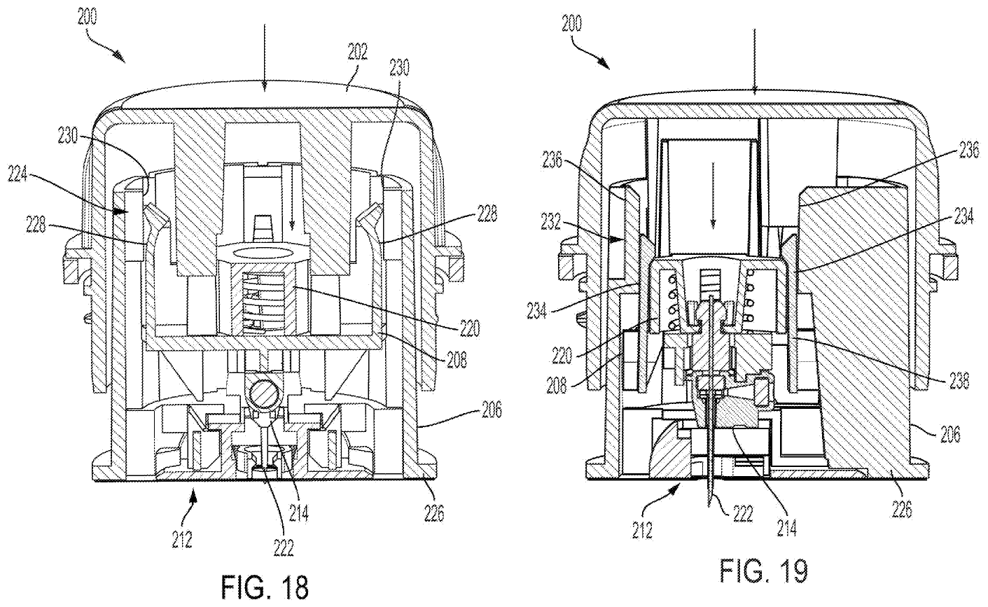

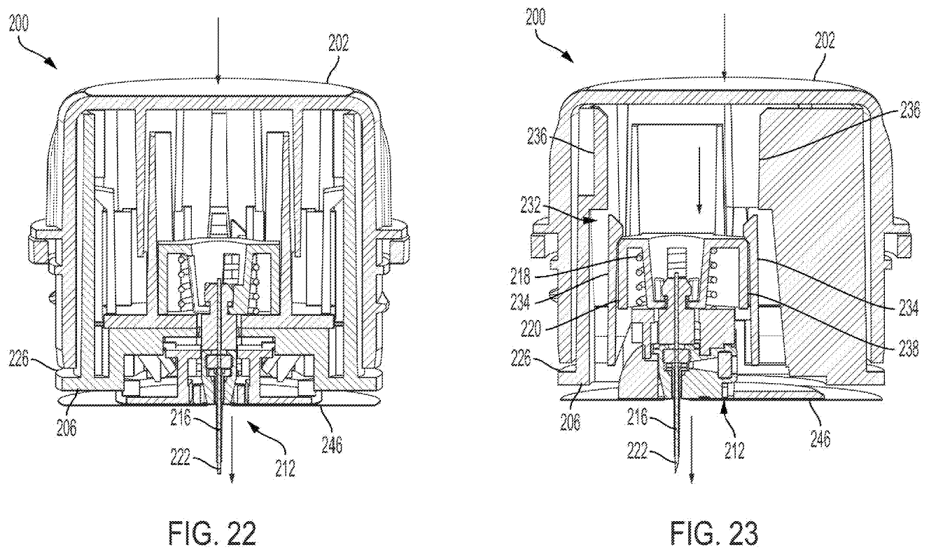

FIGS. 18-23 illustrate the method of using the inserter device 200 to attach the infusion head assembly 212 to the skin of the patient. First, when the inserter device 200 is in the ready state, the base 206 is held against the skin of the patient (not shown) such that the distal end 226 of the base 206 contacts the skin of the patient. Next and referring to FIG. 18, a user (for example, the patient, a medical practitioner, or another person) applies a compressive force to the actuator 202 such that the actuator 202 and the push plate 208 are urged toward the base 206 and the skin of the patient. If the force is less than a threshold (for example, 10 Newtons), the insertion guard 224 holds the push plate 208, the insertion needle 222, and the cannula assembly 214 in the stowed configuration. However and as shown in FIGS. 18 and 19, if the force is equal to or greater than the threshold, the insertion guard 224 permits the push plate 208, the insertion needle assembly 220, and the cannula assembly 214 to move from the stowed configuration toward an insertion configuration.

Illustratively, the insertion guard 224 includes one or more flexible snaps or arms 228 (more specifically, two arms 228) of the push plate 208 that each contact a ledge 230 of the base 206. When the force is equal to or greater than the threshold, the arms 228 deflect and slide over the ledges 230, as shown in FIG. 18, to permit the device 200 to move from the stowed configuration toward the insertion configuration. In other embodiments, the insertion guard 224 may take other forms.

With specific reference to FIG. 19, when the push plate 208 moves from the stowed configuration toward the insertion configuration, the needle assembly 220 moves with the push plate 208 due to the presence of a retraction guard 232. Illustratively, the retraction guard 232 includes one or more flexible snaps or arms 234 (more specifically, two arms 234) of the push plate 208 that each slidably contact a retainer wall 236 of the base 206. The retainer walls 236 hold the arms 228 in contact with a needle carrier 238 of the needle assembly 220 and thereby cause the needle assembly 220 to move with the push plate 208 from the stowed configuration toward the insertion configuration. In other embodiments, the retraction guard 232 may take other forms.

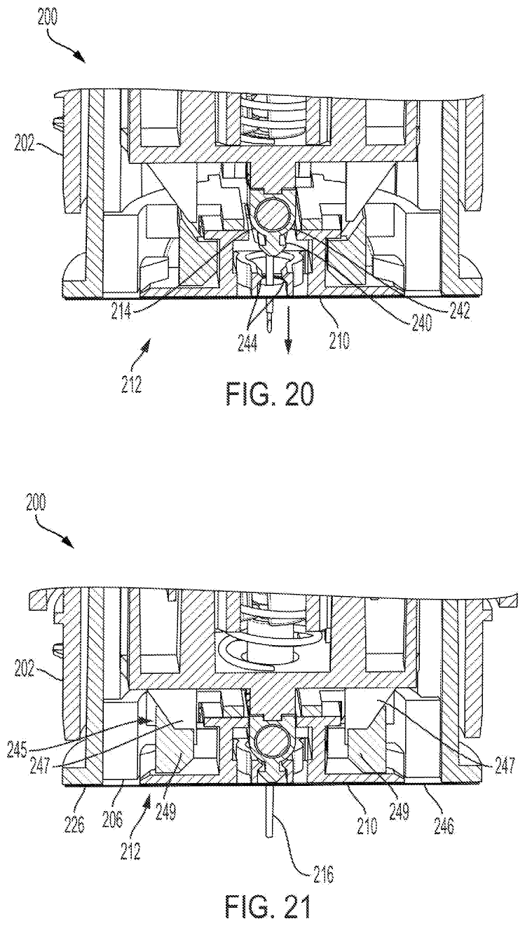

As shown in FIG. 20, continued application of a compressive force to the actuator 202 causes the cannula assembly 214 to be coupled to the infusion head 210. More specifically, a barb 240 of a cannula hub 242 of the cannula assembly 214 couples to one or more snaps or arms 244 (for example, two arms 244) of the infusion head 210.

Referring specifically to FIG. 21, continued application of a compressive force to the actuator 202 causes disengagement of a deployment guard 245 which initially held the infusion head 210 to the base 206. More specifically, the deployment guard 245 includes one or more ramps 247 (for example, two ramps 247) of the push plate 208 that each engage and displace one or more ramps 249 of the base 206 to release the infusion head 210 from the base 206.

As shown in FIGS. 21-23, continued application of a compressive force to the actuator 202 causes the inserter device 200 to reach the insertion configuration. In the insertion configuration, the insertion needle 222 and the infusion cannula 216 extend outwardly from the distal end 226 of the base 206. As a result, the insertion needle 222 pierces the skin of the patient and the infusion cannula 216 enters the subcutaneous space of the patient. In the insertion configuration, the device 200 also secures the infusion head assembly 212 to the skin via an adhesive patch or surface 246 carried on the distal surface of the infusion head 210. In some embodiments, the adhesive surface 246 is the only adhesive surface of the device 200. More specifically and advantageously, the device 200 lacks a second adhesive surface or layer that is initially secured to the skin of the patient and subsequently secured to the infusion head assembly 212 via the adhesive surface 246.

Referring specifically to FIG. 23, after reaching the insertion configuration the retraction guard 232 disengages and permits the retraction mechanism 218 to automatically move the needle assembly 220 from the insertion configuration to a retracted configuration in the base 206. More specifically, the arms 234 of the retraction guard 232 disengage the retainer walls 236 of the base 206, and the arms 234 are deflected outwardly by expansion of the retraction mechanism 218 and proximal movement of the needle assembly 220 relative to the base 206 and proximal movement not shown). As a result, the needle carrier 238 slides over the arms 234, and the needle assembly 220 moves from the insertion configuration to the retracted configuration (not shown, although similar to the retracted configuration of the device 100). The infusion head assembly 212, however, remains attached to the skin of the patient.

Although not specifically illustrated, the inserter device 200 is then moved away from the skin of the patient to leave the infusion head assembly 212 secured to the skin of the patient. Finally, the empty inserter device 200 may be discarded, and the infusion head assembly 212 may be used to deliver a drug to the patient.

Inserter devices according to the present disclosure may be modified in various other manners. For example, inserter devices according to the present disclosure may lack deployment guards, such as the deployment guard 140. In some embodiments of the shift head inserter device 100, a support post 144 of the cap 104 (see FIGS. 4 and 5) may instead secure the infusion head assembly 110 before the cap 104 is detached. In some embodiments of the shift fluid path inserter device 200, the cap 204 itself may secure the infusion head assembly 212 before the cap 204 is detached. In both cases, friction force between the insertion needle and the infusion cannula may also secure the infusion head assembly before attachment to the patient.

Inserter devices according to the present disclosure are illustrated with infusion head assemblies having cannulas that are offset relative to the center of the inserter device. However, and as another exemplary modification, inserter devices according to the present disclosure can include other types of infusion head assemblies, including infusion head assemblies having cannulas that are aligned with the center of the inserter device.

Inserter devices according to the present disclosure may provide various additional advantages over other inserter devices. For example, inserter devices according to the present disclosure may be relatively easy to manufacture and relatively inexpensive. More specifically, inserter devices according to the present disclosure have relatively few components, and may be manufactured through use of simple open/shut molds and automated assembly methods.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An inserter device comprising:
a base;
an actuator movably coupled to the base;
a push plate coupled to the actuator and movable with the actuator relative to the base;
an infusion cannula detachably coupled to the push plate and configured to be inserted into a subcutaneous space of a patient;
an insertion needle coupled to the push plate and the infusion cannula; and
a guard (1) retaining the push plate, the insertion needle, and the infusion cannula in a stowed configuration in the base when a force applied to the actuator is less than a threshold; and (2) permitting the push plate, the insertion needle, and the infusion cannula to move from the stowed configuration to an insertion configuration in which the infusion cannula and the insertion needle extend from the base when the force applied to the actuator is equal to or greater than the threshold.

2. The inserter device of claim 1, wherein the guard comprises an arm, the arm slidably contacting a ledge to (1) retain the push plate, the insertion needle, and the infusion cannula in the stowed configuration when the force applied to the actuator is less than the threshold; and (2) permit the push plate, the insertion needle, and the infusion cannula to move from the stowed configuration to the insertion configuration when the force applied to the actuator is equal to or greater than the threshold.

3. The inserter device of claim 2, wherein the push plate comprises the arm and the base comprises the ledge.

4. The inserter device of claim 1, wherein the guard comprises a plurality of arms, each of the plurality of arms slidably contacting a ledge to (1) retain the push plate, the insertion needle, and the infusion cannula in the stowed configuration when the force applied to the actuator is less than the threshold; and (2) permit the push plate, the insertion needle, and the infusion cannula to move from the stowed configuration to the insertion configuration when the force applied to the actuator is equal to or greater than the threshold.

5. The inserter device of claim 1, further comprising a retraction mechanism coupled to the push plate and the insertion needle, the retraction mechanism automatically pushing the insertion needle to a retracted configuration in the base after the insertion needle moves from the stowed configuration to the insertion configuration.

6. The inserter device of claim 5, wherein the retraction mechanism pushes the insertion needle away from the base when pushing the insertion needle to the retracted configuration.

7. The inserter device of claim 5, wherein the guard is an insertion guard, and further comprising a retraction guard, the retraction guard inhibiting the retraction mechanism from pushing the insertion needle to the retracted configuration before the infusion cannula and the insertion needle move to the insertion configuration.

8. The inserter device of claim 7, wherein the guard is an insertion guard, and further comprising a deployment guard, the deployment guard inhibiting the infusion cannula from detaching from the push plate before the infusion cannula and the insertion needle move from the stowed configuration to the insertion configuration.

9. An inserter device comprising:
a base;
an actuator movably coupled to the base;
a push plate coupled to the actuator and movable with the actuator relative to the base;
an infusion cannula detachably coupled to the push plate and configured to be inserted into a subcutaneous space of a patient;
an insertion needle coupled to the push plate and the infusion cannula; and
a guard (1) inhibiting movement of the actuator, the push plate, the insertion needle, and the infusion cannula relative to the base when a force applied to the actuator is less than a threshold; and (2) permitting movement of the actuator, the push plate, the insertion needle, and the infusion cannula relative to the base when the force applied to the actuator is equal to or greater than the threshold, the push plate and the insertion needle thereby inserting the infusion cannula into the subcutaneous space of the patient.

10. The inserter device of claim 9, wherein the guard comprises an arm, the arm slidably contacting a ledge to (1) inhibit movement of the actuator, the push plate, the insertion needle, and the infusion cannula relative to the base when the force applied to the actuator is less than the threshold; and (2) permit movement of the actuator, the push plate, the insertion needle, and the infusion cannula relative to the base when the force applied to the actuator is equal to or greater than the threshold.

11. The inserter device of claim 10, wherein the push plate comprises the arm and the base comprises the ledge.

12. The inserter device of claim 9, wherein the guard comprises a plurality of arms, each of the plurality of arms slidably contacting a ledge to (1) inhibit movement of the actuator, the push plate, the insertion needle, and the infusion cannula relative to the base when the force applied to the actuator is less than the threshold; and (2) permit movement of the actuator, the push plate, the insertion needle, and the infusion cannula relative to the base when the force applied to the actuator is equal to or greater than the threshold.

13. The inserter device of claim 9, wherein the insertion needle is detachably coupled to the push plate.

14. The inserter device of claim 13, further comprising a retraction mechanism coupled to the push plate and the insertion needle, the retraction mechanism automatically pushing the insertion needle away from the push plate and into the base after the insertion needle inserts the infusion cannula into the subcutaneous space of the patient.

15. The inserter device of claim 14, wherein the guard is an insertion guard, and further comprising a retraction guard, the retraction guard inhibiting the retraction mechanism from pushing the insertion needle away from the push plate before the insertion needle inserts the infusion cannula into the subcutaneous space of the patient.

16. The inserter device of claim 15, wherein the guard is an insertion guard, and further comprising a deployment guard, the deployment guard inhibiting the infusion cannula from detaching from the push plate before the insertion needle inserts the infusion cannula into the subcutaneous space of the patient.
